(19) United States
(12) Patent Application Publication
(10) Pub. No.: US 2015/0265654 A1
(43) Pub. Date: Sep. 24, 2015

(54) METHOD FOR THE TREATMENT OF DAMAGED TISSUE

(71) Applicant: Patrick J. Casey, Kumeu (NZ)

(72) Inventor: Patrick J. Casey, Kumeu (NZ)

(21) Appl. No.: 14/662,894

(22) Filed: Mar. 19, 2015

Related U.S. Application Data
(60) Provisional application No. 61/968,117, filed on Mar. 20, 2014.

(51) Int. Cl. A61K 35/34; C12N 5/077

(52) U.S. Cl. CPC A61K 35/34; C12N 5/0658

(57) ABSTRACT

The present invention provides a method for treating a subject animal having a damaged tissue. In one aspect of the invention, the method includes the steps of harvesting a tissue sample from a tissue source; growing a plurality of cells from the tissue sample in a culture system; obtaining a sample of the grown cells from the culture system; and introducing the sample of the grown cells into the subject animal for aiding in repair of the damaged tissue; wherein the introducing step includes introducing the sample of the grown cells into a region of the subject animal not including the damaged tissue.

### STATEMENT REGARDING FEDERALLY SPONSORED RESEARCH OR DEVELOPMENT

Not Applicable.

### BACKGROUND OF THE INVENTION

Damaged, diseased tissue, and/or otherwise compromised tissue are common ailments in many animals including mammals. Treatment options are limited and often ineffective. Accordingly, it would be desirable to develop improved methodologies for treating an animal, and preferably a mammal, having damaged, diseased or otherwise compromised tissue.

### BRIEF SUMMARY OF THE INVENTION

The present invention provides a method for treating a subject animal having a damaged tissue. In one aspect of the invention, the method includes the steps of harvesting a tissue sample from a tissue source; growing a plurality of cells from the tissue sample in a culture system; obtaining a sample of the grown cells from the culture system; and introducing the sample of the grown cells into the subject animal for aiding in repair of the damaged tissue; wherein the introducing step includes introducing the sample of the grown cells into a region of the subject animal not including the damaged tissue.

In one embodiment, the step of introducing the sample of the grown cells into the subject animal includes injecting the sample of the grown cells into the subject animal. In other embodiments, the injecting step includes injecting the sample of the grown cells into the subject animal using an injection application selected from the group consisting of an intravenous injection, a subcutaneous injection, an intramuscular injection, an intra-cerebral spinal cord injection, an intraocular injection, an intranasal injection, an intra-articular injection, an intra dermal injection and a sub-dermal injection.

In another embodiment, the step of growing the cells from the tissue sample in the culture system includes breaking the tissue sample into a plurality of tissue fragments; placing the tissue fragments into a culture vessel; starving the tissue fragments of nutrients for a period of nutrient deprivation; and following the period of nutrient deprivation, supplying the tissue fragments with nutrients. In yet another embodiment, the step of growing the cells includes adding media sufficient to maintain a moist environment in the culture vessel without contacting the tissue fragments with the media.

In other embodiments, the step of breaking the tissue sample into the tissue fragments includes at least one of dissecting the tissue fragments, chemically digesting the tissue fragments, and physically digesting the tissue fragments. In one embodiment, the step of breaking the tissue sample into the tissue fragments includes digesting the tissue fragments with Trypsin and ethylenediaminetetraacetic acid (EDTA).

In yet another embodiment, the step of obtaining a sample of the grown cells from the subject culture system includes removing the cells by incubating the cells with Trypsin/EDTA.

In one embodiment, the tissue source is the subject animal. In another embodiment, the subject animal is a mammal and in a further embodiment, the mammal is a human.

In an additional embodiment, the step of introducing the sample of the grown cells into the subject animal for aiding in repair of the damaged tissue comprises transporting the sample at an ambient temperature of between 12° C. to 30° C. from a point where the sample of grown cells is obtained to the point where the sample of grown cells is introduced into the subject animal.

In yet a further additional embodiment, the method includes the step of obtaining a preservation sample of the grown cells from the culture system; and cryo-preserving the preservation sample for future culture.

### BRIEF DESCRIPTION OF THE SEVERAL VIEWS OF THE DRAWINGS

FIG. 1 is a drawing showing the steps of the invention, according to one embodiment of the invention.

### DETAILED DESCRIPTION OF THE INVENTION

This application incorporates by reference the following published patent applications: US 2008/0311085, 2009/0010898, 2009/0004154, and 2009/0004155.

The present invention generally relates to a method for treating a subject animal and preferably a mammal where at least some portion of the tissue in the animal is damaged, diseased, or otherwise compromised. It has been surprisingly found that grown cells can be introduced systemically into an individual into non-damaged areas where they will migrate to the area of damage through a tropism. While the present invention is discussed with respect to mammals, the present invention can be applied to all members of the animal kingdom.

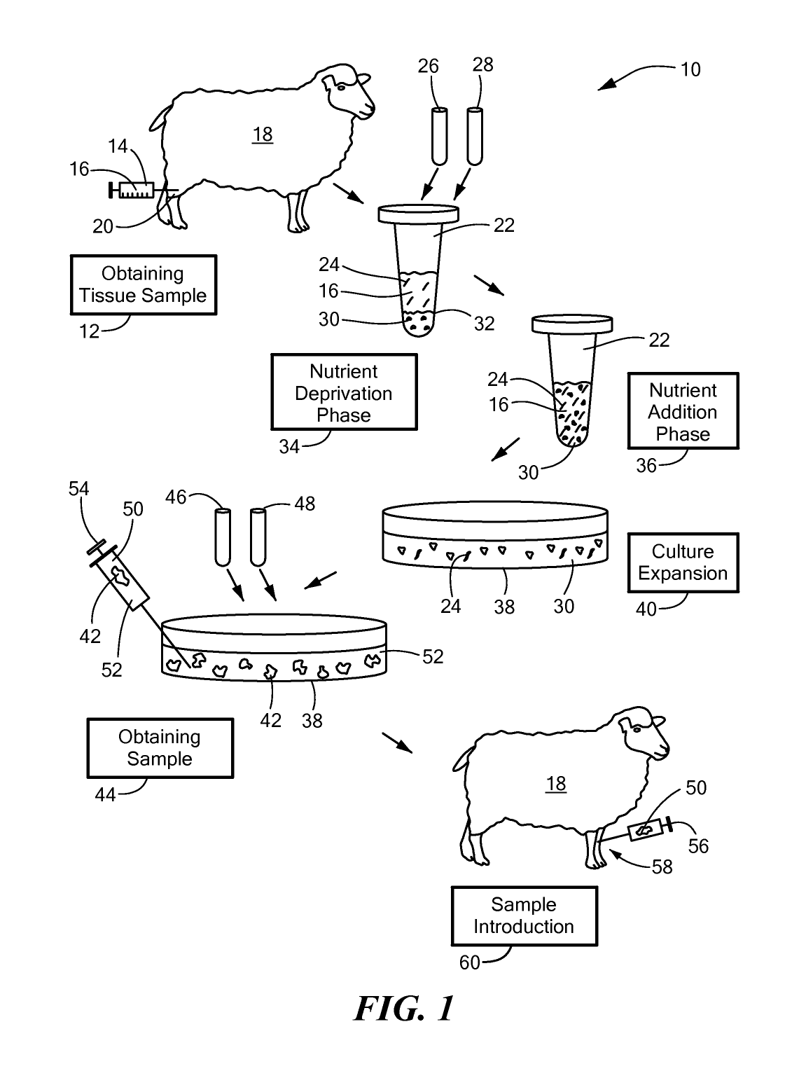

An embodiment 10 of the method is exemplified in FIG. 1. For the purpose of this application, the term “damaged tissue” as used herein refers to damaged, diseased and/or otherwise compromised tissue including, for example, cells which have been severely damaged (including apoptosis) or in fact have suffered cell death (necrosis). The method involves harvesting a tissue sample from a tissue source, as shown in step 12 of FIG. 1. The tissue samples are harvested from mammals having the necessary tissue sources using minimally invasive injection or biopsy techniques known to those of ordinary skill in the art in such a manner as to ensure that minimal damage is caused to the surrounding tissue while obtaining a sufficient amount of tissue sample required for growing the necessary supply of grown cells. A syringe 14 can be used to harvest the tissue sample 16 from the subject animal 18 in a region of non-damaged tissue 20, as shown in step 12 of FIG. 1.

The harvested tissue sample 16 is transported to a laboratory preferably at ambient temperatures, such as, for example, 12° C. to 30° C., and preferably 20° C. to 24° C., and more preferably at 22° C. for in vitro growth of cells. The sample should be transported in culture media that contains protein (for example Fetal Calf serum 5-19%). The harvested tissue sample 16 is placed in a unique culture system including, for example, a culture vessel such as a culture flask 22. In one embodiment, the tissue sample 16 is broken down into tissue fragments 24 to enhance cellular growth. Tissue fragmentation is accomplished with any suitable technique known to those of ordinary skill in the art, including but not limited to dissection, chemical digestion, and physical digestion. In one embodiment, a proteolytic enzyme such as, for example, Trypsin 26, in conjunction with a calcium stabilizing agent such as, for example, EDTA 28, is used to break down junctions between tissues to form tissue fragments 24, as shown in FIG. 1.

Media 30 is supplied to the culture system for moisture and nutrients. In a preferred embodiment, media 30 is initially supplied to the culture system in a compartmentalized fashion such as in a separate compartment 32 in order to ensure that the tissue fragments 24 are kept moist but deprived of physical contact with the media. The moisture induces the tissue fragments to adhere, attach and/or “stick” to the culture vessel which is necessary for the cells to grow and divide. The tissue fragments 24 undergo a period or phase of nutrient deprivation 34 because they are initially kept isolated from the media, as shown in FIG. 1. The nutrient deprivation phase 34 can last from 12 to 76 hours, and preferably from 24 hours to 60 hours, and more preferably from 36 hours to 48 hours, and most preferably for approximately 48 hours until the cells slow down into the desired stressed state.

Prior to death of the cells, the tissue sample 16 including tissue fragments 24 are physically contacted with media 30 during a nutrient addition phase 36 to provide nutrients to the cells. Media 30 is physically contacted with and/or added to the tissue fragments 24 through methods known to those of ordinary skill in the art. The stressed cells respond to the introduction of nutrients in the media 30 by entering into a pre-mitotic state and eventually a mitotic state and dividing. The media 30 is refreshed at periods known to those of ordinary skill in the art, at for example, 1 to 4 day intervals and more preferably and 2-3 day intervals. The conditions for cellular growth including, for example, the type and amount of media added to the culture system as well as the length of the nutrient addition phase 36 depend upon the initial amount of tissue sample, and the desired amount of cell growth given the type of cells being grown. For example, tenocytes divide on average 5 days post media addition, articular cartilage cells divide approximately every 14 days, and islet cells approximately every 9 days.

The culture system is maintained until a predetermined amount of cells are grown after a preselected period of time. Contact between normal non-cancerous cells inhibits growth, however. When the cells are 60-90% confluent, and preferably 70-80% confluent, the cells are pulled apart, re-suspended, and plated in another culture system 38 to maintain cell growth in the mitotic state. This step is shown as the culture expansion phase 40 in FIG. 1.

After the appropriate number of cell division cycles have been completed, a sample 50 including a suspension 52 of grown cells 42 is obtained, as shown in step 44 in FIG. 1.

Preferably, the grown cells 42 are obtained from the culture system using a Trypsin/EDTA 46 incubation technique and washed using phosphate buffered saline 48. Typically the cells which have been pulled apart in the digestion process remain apart for up to 48 hours. The sample 50 including the suspension 52 of grown cells 42 is centrifuged and the centrifuged grown cells are loaded into an appropriate transport vessel, such as, for a non-limiting example, a syringe 54. Other non-limiting examples of transport vessels include sterile Falcon ™ tubes and/or test tubes with lids. Transport of the grown cells occurs at ambient temperatures such as, for example, 12° C. to 30° C., and preferably 20° C. to 24° C., and more preferably at 22° C. to maintain the cells in a metabolically active state. The sample 50 of the grown cells 42 can be shipped for a period of up to 48 hours.

The sample 50 including the grown cells 42 is loaded into an application device such as, for example, a syringe 56, for introduction into the subject animal. Although the transport vessel can be used as the application device, it is preferable to use an application device which is different from the transport vessel. The grown cells which are provided to the subject animal are of the correct type to avoid rejection by the subject mammal because the cells are from the same mammal such the cells are recognized by the individual mammal as “self” and not rejected by the mammal's immune system.

The sample 50 of grown cells 42 is introduced into the same individual who provided the tissue. Thus, the transplantation is referred to as Auto-transplantation. FIG. 1 shows the cell sample 50 being introduced into the same animal 18 which provided the tissue sample for cell. The grown cells can be injected directly into the area of tissue damage. Alternatively, and surprisingly, the grown cells can be injected systemically into the individual into non-damaged areas where they will migrate to the area of damage through a tropism. Injection into non-damaged regions is preferable where direct injection into damaged tissue might cause trauma, such as, for a non-limiting example, direct injection into a beating heart. In FIG. 1, the sample 50 including the grown cells 42 is introduced into the tissue provider 18 in an area 58 which does not include the damaged tissue. The step is described as sample introduction 60 in FIG. 1.

The new cells grown in vitro occupy the space of damage in the receiving individual and establish a new cell colony with natural incorporation into the surrounding tissue. The cells enact repair by filling the surrounding tissue and in some cases by making a new cellular matrix, such as, for non-limiting examples, new tendon and/or ligament structures.

The grown cells can be injected into the subject mammal using a variety of injection application techniques known to those of ordinary skill in the art including, for non-limiting examples, intravenous injection, subcutaneous injection, intramuscular injection, intra-cerebral spinal cord injection, intraocular injection, intranasal injection, intra-articular injection, intra dermal injection and sub-dermal injection. The application technique is selected based on the type of tissue treatment required. In one exemplary embodiment, myocardial cells can be injected intravenously so they can migrate to the damaged myocardial tissue without causing damage from direct injection into the heart. Upon injection, the grown cells migrate to the area of the damaged tissue and aid in its repair.

It is routine to cryo-preserve (−196° C.) a sample of the grown cells so that they may be used for further culture for that individual at some later time.

The number of and the manner in which the cells are harvested are selected based on minimizing tissue damage while obtaining the number of cells necessary to be representative of the target tissue. As little as 50 cells can be harvested. The sample of grown cells introduced into the subject animal can include numbers of cells which are greater that the number of harvested cells by orders of magnitude. For example, a sample of grown cells re-introduced into the subject mammal can include millions and even billions of cells. The number of cells to be transplanted is determined clinically according to methods known to those of ordinary skill in the art and depends upon the amount of tissue damage and the subsequent number of cells required to repair the damage and regenerate the tissue and/or organ. When the tissue sample is harvested from the animal having the damaged tissue, the tissue sample is harvested in such a manner so as to avoid or minimize damage or other trauma to the already damaged tissue. Similarly, the application technique for the injection of the grown cells is selected to prevent or minimize damage or other trauma to the surrounding damaged tissue.

The method of the invention can be used to treat various types of damaged tissue, such as, for example, connective tissue, articular cartilage tissue, pancreatic tissue, myocardium tissue. Connective tissue for treatment can include, for example, tissue containing tendon or ligament cells. In a non-limiting example, if an individual mammal has a damaged piece of tendon and/or ligament tissue, then a small biopsy of tendon and/or ligament tissue can be made adjacent to the damage or from an area of little tissue impact. The sample can be transported to the laboratory and uniquely grown and then transported back to the individual for application into either the site or injury or non-injured sites.

While specific embodiments of the present invention have been described, it should be noted that various modifications thereto can be made without departing from the spirit and scope of the invention as defined in the appended claims.

What is claimed is:

1. A method for treating a subject animal having a damaged tissue, the method comprising:
harvesting a tissue sample from the subject animal;
growing a plurality of cells from the tissue sample in a culture system;
obtaining a sample of the grown cells from the culture system; and
introducing the sample of the grown cells into the subject animal for aiding in repair of the damaged tissue;
wherein the introducing step comprises introducing the sample of the grown cells into a region of the subject animal not including the damaged tissue.

2. The method of claim 1, wherein introducing step comprises injecting the sample of the grown cells into the subject animal.

3. The method of claim 2, wherein the injecting step comprises injecting the sample of the grown cells using an injection application selected from the group consisting of an intravenous injection, a subcutaneous injection, an intramuscular injection, an intra-cerebral spinal cord injection, an intraocular injection, an intranasal injection, an intra-articular injection, an intra dermal injection and a sub-dermal injection.

4. The method of claim 1, wherein the step of growing the plurality of cells from the tissue sample in a culture system comprises:
breaking the tissue sample into a plurality of tissue fragments;
placing the tissue fragments into a culture vessel;
inducing cells of the tissue fragments to attach to the culture vessel while starving the tissue fragments of nutrients for a period of nutrient deprivation; and
following the period of nutrient deprivation, supplying the tissue fragments with nutrients.

5. The method of claim 4, wherein the step of inducing cells of the tissue fragments to attach to the culture vessel comprises:
adding media sufficient to maintain a moist environment in the culture vessel without contacting the tissue fragments with the media.

6. The method of claim 4, wherein the step of breaking the tissue sample into the tissue fragments comprises at least one of methods of dissecting the tissue fragments, chemically digesting the tissue fragments, and physically digesting the tissue fragments.

7. The method of claim 4, wherein the step of breaking the tissue sample into the tissue fragments comprises digesting the tissue sample with trypsin and EDTA.

8. The method of claim 1, wherein the step of obtaining the sample of the grown cells from the culture system comprises removing the cells by incubating the cells with Trypsin and EDTA.

9. The method of claim 1, wherein the subject animal is a mammal.

10. The method of claim 10, wherein the mammal is a human.

11. The method of claim 1, further comprising the steps of
prior to the introducing step, adding a media including a protein to the sample of grown cells; and
transporting the sample of grown cells at an ambient temperature of between 12° C. to 30° C. from a point where the sample of grown cells is obtained to the point where the sample of grown cells is introduced into the subject animal.

12. The method of claim 1, further comprising the steps of
obtaining a preservation sample of the grown cells from the culture system; and
cryo-preserving the preservation sample for future culture.
